(19) United States
(12) Patent Application Publication
(10) Pub. No.: US 2016/0303153 A1
(43) Pub. Date: Oct. 20, 2016

(54) Topical Lotion Composition Comprising Ivermectin

(71) Applicant: Gavis Pharmaceuticals, Somerset, NJ (US)

(72) Inventor: Bala Chandran Nayar, Somerset, NJ (US)

(73) Assignee: Gavis Pharmaceuticals, Somerset, NJ (US)

(21) Appl. No.: 14/687,778

(22) Filed: Apr. 15, 2015

(51) Int. Cl. A61K 31/7048; A61K 9/00; A61K 47/06; A61K 47/10

(52) U.S. Cl. CPC A61K 31/7048; A61K 47/10; A61K 9/0014; A61K 47/06

(57) ABSTRACT

A topical lotion composition of ivermectin is provided. The topical lotion composition contains a pharmaceutically acceptable alcohol and a pharmaceutically acceptable gelling agent. The composition is essentially devoid of any surfactant.

### BACKGROUND OF THE INVENTION

(a) Field of the Invention

The present invention is directed to a topical lotion composition comprising ivermectin. The composition is particularly in the form of an aqueous lotion composition comprising a pharmaceutically acceptable alcohol and a gelling agent. The invention is also directed to use of said topical lotion composition of ivermectin for the treatment of rosacea, and particularly, for the treatment of inflammatory lesions of rosacea.

(b) Description of the Related Art

Ivermectin is a mixture of two compounds belonging to the class of avermectins, 5-O-demethyl-22, 23-dihydroavermectin A₁ₐ and 5-O-demethyl-22, 23-dihydroavermectin A_{1b}. They are also known as 22, 23-dihydroavermectin B₁ₐ and 22, 23-dihydroavermectin B_{1b}. Ivermectin is a mixture containing at least 90% 5-O-demethyl-22,23-dihydroaverrnectin A₁ₐ and less than 10% 5-O-demethyl-25-de(1-methylpropyl)-22,23-dihyro -25-(1-methylethyl) avermectin A₁ₐ, generally referred to as 22,23-dihydro avermectin B₁ₐ and B_{1b}, or H₂B₁ₐ and H₂B_{1b}, respectively. Ivermectin contains at least 80% of 22, 23-dihydroavermectin B₁ₐ and less than 20% of 22, 23-dihydroavermectin B_{1b}. The structural formulas are:

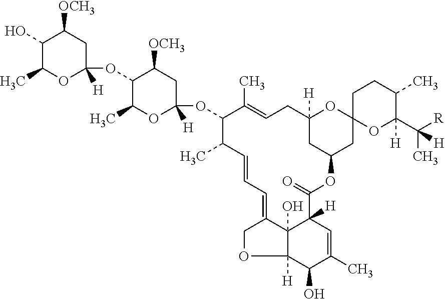

Component B₁ₐR═C₂H₅ and Component B_{1b}R═CH₃

This active agent forms part of the class of avermectins, a group of macrocyclic lactones produced by the bacterium Streptomyces avermitilis (Reynolds J E F (Ed) (1993) Martindale, The Extra Pharmacopoeia. 29th Edition. Pharmaceutical Press, London). Avermectins include in particular ivermectin, invermectin, avermectin, abamectin, doramectin, eprinomectin and selamectin.

In the middle of the 1980s, ivermectin was presented as a broad-spectrum anti-parasitic medicinal product for veterinary use (W. C. Campbell, et al., (1983). Ivermectin: a potent new anti-parasitic agent, Science, 221, 823-828). It is effective against most common intestinal worms (except tapeworms), most acarids and some lice. It in particular exhibits considerable affinity for the glutamate-dependent chloride channels present in invertebrate nerve cells and muscle cells. Its binding to these channels promotes an increase in membrane permeability to chloride ions, resulting in hyperpolarization of the nerve or muscle cell. Neuromuscular paralysis which can lead to the death of certain parasites results therefrom. Ivermectin also interacts with other ligand-dependent chloride channels, such as those involving the neuromediator GABA (gamma-aminobutyric acid).

Ivermectin is more particularly an anthelmintic. It has been administered in humans in the treatment of onchocerciasis caused by onchocerca volvulus, of gastrointestinal strongyloidiasis (anguillulosis) (product Stomectol®) and of human scabies (Meinking T Let al., N. Engl. J. Med., 1995 Jul. 6; 333(1): 26-30, The treatment of scabies with ivermectin) and also in the treatment of microfilaremia diagnosed or suspected in individuals suffering from lymphatic filariosis due to Wuchereria bancrofti.

U.S. Pat. No. 4,199,569, discloses ivermectin as a semisynthetic, anthelmintic agent derived from the avermectins, a class of highly active broad-spectrum anti-parasitic agents isolated from the fermentation products of Streptomyces avermitilis.

U.S. Pat. Nos. 6,399,652; 6,399,651 and 6,319,945 disclose methods of treating skin disorders via application of topical formulations containing ivermectin to treat acne vulgaris (the '652 patent), a variety of dermatoses (e.g., transient acantholytic dermatitis, acne miliaris necrotica, acne varioliformis, perioral dermatitis, and acneiform eruptions; the '651 patent) and seborrheic dermatitis (the '945 patent). Further, U.S. Pat. Nos. 6,399,651; 6,399,652 and 7,897,559 disclose dermatological compositions containing an avermectin compound for treating dermatoses including transient acantholytic dermatitis, acne miliaris necrotica, acne varioliformis, perioral dermatitis, and acneiform eruptions. The patents exemplify a lotion formulation of ivermectin comprising Cetaphil® moisturizing lotion as base. The formulation contains a large number of additives including, viz. purified water, glycerin, hydrogenated polyisobutene, cetearyl alcohol and ceteareth-20, macadamia nut oil, dimethicone, tocopheryl acetate, stearoxtrimethylsilane and stearyl alcohol, panthenol, famesol, benzyl alcohol, phenoxyethanol, acrylates/C10-30 alkyl acrylate crosspolymer, sodium hydroxide, and citric acid.

PCT Application Pub. No. WO 2004/093886 discloses the use of ivermectin for the manufacture of a topical pharmaceutical composition intended for human use. More particularly, this application discloses the use of topical pharmaceutical compositions intended for human use comprising ivermectin for the treatment of dermatological conditions such as rosacea, acne vulgaris, seborrhoeic dermatitis, perioral dermatitis, acneform rash, transient acantholytic dermatitis and acne miliaris necrotica.

U.S. Pat. No. 5,952,372 describes a method for treating rosacea using ivermectin orally or topically in order to reduce and eliminate the parasite Demodex folliculorum present on the skin of patients.

U.S. Pat. Nos. 6,103,248 and 8,791,153 disclose a topical preparation for applying to scalp hair for the treatment of head lice containing a surfactant to allow the topical preparation to be washed out of the hair.

U.S. Pat. No. 8,912,151 discloses a composition of macrocyclic lactone compounds including avermectin compounds for treating hemorrhoids. A lotion formulation of ivermectin comprising Cetaphil® moisturizing lotion as a base is exemplified in the patent. The formulation also contains a large number of additives.

The low compatibility of ivermectin with many excipients (N. O. Shaw, M. M. de Villiers and A. P. Lötter, Pharmazie, 54 (1999) 5, 372-376 Preformulation stability screening of ivermectin with non-ionic emulsion excipients), and its low solubility in water mean that pharmaceutical compositions containing ivermectin generally require either the addition of a large number of additives which make it possible to obtain stable compositions, which has the effect of increasing the risk of allergies, or to be formulated with anhydrous excipients. The anhydrous compositions encountered conventionally have the disadvantage of a greasy feel and therefore of an appearance that is not very cosmetic, which may be responsible for a decrease in patient compliance. In addition, by virtue of the low stability of ivermectin in water, the shelf life of aqueous compositions containing ivermectin is generally shorter than that of anhydrous compositions containing ivermectin.

Various methods have been provided in the prior art to stabilize ivermectin in aqueous compositions and overcome its chemically instability on contact with water.

European Pat. No. EP 0,045,655 B1 proposes forming micelles of surfactants which surround the ivermectin in order to protect it against water; other applications, such as PCT Application Pub. Nos. WO 01/60380 and WO 97/26895, propose using aqueous solvents for active agents, such as N-methyl-2-pyrrolidone. Finally, PCT Application Pub. Nos. WO 2004/093886 and WO 2005/089806 describe emulsions comprising an oily phase and an aqueous phase, said aqueous phase comprises a micellar active phase containing ivermectin.

There still exists a need for topical pharmaceutical compositions containing ivermectin which are completely suited to the pathological condition, and specifically to sensitive skin, which is industrially acceptable, i.e., the formulation of which is physically stable (without phase separation), and chemically stable (without modification of the stability of the active agent) and which optimizes the penetration of ivermectin into the skin. Therefore, there still exists a need to provide an alternative, chemically and physically stable and effective topical formulation, particularly an aqueous lotion formulation of ivermectin which contains relatively less number of additives (or have minimum additive load), exhibit good physical and chemical stability and good tolerance on the skin.

### SUMMARY OF THE INVENTION

The present invention provides a topical lotion composition of ivermectin. The composition is in the form of an aqueous lotion comprising a pharmaceutically acceptable alcohol and a pharmaceutically acceptable gelling agent. Essentially, the lotion is devoid of surfactants.

In one aspect, the invention provides a topical aqueous lotion composition comprising:
(a) Ivermectin;
(b) one or more pharmaceutically acceptable alcohols;
(c) one or more pharmaceutically acceptable gelling agents; and
(d) water;

wherein said lotion composition is devoid of a surfactant.

In another aspect, the invention provides a topical aqueous lotion composition comprising:
(a) Ivermectin;
(b) one or more pharmaceutically acceptable alcohols selected from the group consisting of methanol, benzyl alcohol, ethyl alcohol and isopropyl alcohol;
(c) one or more pharmaceutically acceptable gelling agents selected from the group consisting of hydroxypropyl cellulose, hydroxypropyl methylcellulose, carrageenans, microcrystalline cellulose, alginates, gellan gum, xanthan gum, veegum, hydroxyethyethylcellulose, guar gum, and carbomers; and
(d) water;
   wherein said lotion composition is devoid of surfactant.

In another aspect, the invention provides a topical aqueous lotion composition consisting essentially of:
(a) Ivermectin;
(b) one or more pharmaceutically acceptable alcohol comprising isopropyl alcohol;
(c) one or more pharmaceutically acceptable gelling agents comprising carbomer; and
(d) water;
   wherein said lotion composition is devoid of a surfactant.

In another aspect, the amount of alcohol in the topical aqueous lotion composition of the invention is at least 35% by weight of the composition.

In another aspect, the invention provides a topical aqueous lotion composition comprising:
(a) Ivermectin in an amount of about 0.01% to about 5% by weight;
(b) one or more pharmaceutically acceptable alcohols in an amount of at least 35% by weight;
(c) one or more pharmaceutically acceptable gelling agents in an amount of about 0.01% to about 5% by weight; and
(d) water;
   wherein said lotion composition is devoid of a surfactant.

In another aspect, a topical aqueous lotion composition of ivermectin of the invention remains chemically and/or physically stable over a period of time of at least 8 weeks.

In another aspect, a topical aqueous lotion composition of ivermectin of the invention further comprises one or more additives selected from the group consisting of flavour enhancers, preserving agents, stabilizers, humidity regulators, pH regulators, osmotic pressure modifiers, UV-A screening agents, UV-B screening agents and antioxidants. Essentially, the topical lotion composition of ivermectin of the invention is devoid of any preserving agents and antioxidants.

In another aspect, the invention also provides a method for the treatment of rosacea, more particularly, for the treatment of inflammatory lesions of rosacea comprising topically applying the topical aqueous lotion composition of ivermectin as substantially described herein above on to the affected skin of the patient in need of such treatment.

Still other aspects and advantages of the invention will be apparent from the following detailed description of the invention.

### DETAILED DESCRIPTION OF THE INVENTION

The invention provides for a topical lotion composition of ivermectin with minimum additives load. It was surprisingly found that the composition according to the invention exhibits good stability and good tolerance on the skin. Particularly, it was found that such a stable composition can be prepared using a limited number of additives. Such formulations are also easy to manufacture on a commercial scale.

The lotion composition of the present invention provides a means for topically delivering ivermectin to a patient over a long residence time on the skin. This is particularly important since ivermectin is a slow acting drug. By providing a long residence time, the cosmetically acceptable lotion formulation allows for a therapeutically effective use of environmentally acceptable low quantities of ivermectin. Additionally, use of low quantities of ivermectin may reduce human systemic absorption of the active ingredient.

The ivermectin according to the invention may contain at least 80% of 22, 23-dihydroavermectin B₁ₐ and less than 20% of 22, 23-dihydroavermectin B_{1b}.

The topical lotion composition of ivermectin according to the invention comprises:
(a) Ivermectin;
(b) one or more pharmaceutically acceptable alcohols;
(c) one or more pharmaceutically acceptable gelling agents; and
(d) water.

The topical lotion composition of the invention is suitable for treating rosacea and more particularly for treating inflammatory lesions of rosacea.

Pharmaceutically acceptable alcohols used in topical lotion compositions of the invention are non-toxic, and do not irritate the skin at the concentrations. The alcohol used in the present invention may be selected from a lower hydrocarbon chain alcohol such as a C₁-C₄ alcohol. In a preferred embodiment the alcohol is chosen from ethanol, 2-propanol, methanol, benzyl alcohol, isopropyl alcohol or n-propanol, and most preferably isopropyl alcohol.

The amount in which pharmaceutically acceptable alcohols are present in the topical lotion composition of the invention is at least 35% by weight of the composition.

Pharmaceutically acceptable gelling agents suitable for use in topical lotion compositions of the invention are those agents which provide viscosity to a formulation such that the formulation can be effectively applied to the infested or affected area, including but not limited to hydroxypropylcellulose (e.g. Klucel H A), hydroxypropyl methylcellulose carrageenans, microcrystalline cellulose, alginates, gellan gum, xanthan gum, veegum, hydroxyethyethyl cellulose, guar gum and carbomers. Carbomer methylcellulose is preferred.

The topical lotion composition according to the invention comprises from 0.001% to 10% of ivermectin by weight relative to the total weight of the composition. Preferably, the composition according to the invention contains from 0.1% to 5% of ivermectin by weight relative to the total weight of the composition.

The amount in which pharmaceutically acceptable gelling agents are present in the topical lotion composition of the invention ranges from about 0.01% to about 5% by weight of the composition.

The composition according to the invention is advantageously a lotion which comprises:
(a) Ivermectin in an amount of about 0.01% to about 5% by weight;
(b) one or more pharmaceutically acceptable alcohols in an amount of at least 35% by weight;
(c) one or more pharmaceutically acceptable gelling agents in an amount of about 0.01% to about 5% by weight; and
(d) water.

The topical lotion composition of the invention contains water ranging from 1% to 65% by weight relative to the total weight of the composition. The water used in the composition according to the invention will preferably be purified water.

The compositions according to the invention are described as stable lotion compositions in that they may exhibit good physical and chemical stability over time, even at a temperature above ambient temperature (for example 45-55° C.).

The ivermectin in the topical lotion composition according to the invention also, surprisingly, may exhibit good chemical stability in the case of pH variation.

The topical lotion composition according to the invention may also contain inert additives or combinations of these additives, such as flavour enhancers; preservatives; stabilizers; humidity regulators; pH regulators; osmotic pressure modifiers; UV-A and UV-B screening agents; and antioxidants. Preferably, the topical lotion composition of ivermectin of the invention is devoid of preserving agents and antioxidants.

These additives may be present in the composition at from 0.001% to 20% by weight relative to the total weight of the composition.

A typical lotion composition according to the invention comprises:
(a) Ivermectin;
(b) one or more pharmaceutically acceptable alcohols comprising isopropyl alcohol;
(c) one or more pharmaceutically acceptable gelling agents comprising carbomer; and
(d) water.

The pH of the composition preferably ranges from 5.0 to 8.0. Verification of the natural pH of the mixture and possible correction with a solution of a neutralizing agent, and also the incorporation of the optional additives, may be carried out, according to their chemical nature, during one of the steps of the method of preparation, described above.

Preparation of the lotion composition of the invention involves the sequential or separate mixing of ivermectin in pharmaceutically acceptable alcohol, the pharmaceutically acceptable gelling agent, and water.

A preferred preparation involves the mixing of ivermectin in isopropyl alcohol, separately mixing carbomer in water and mixing together of the two mixtures to form an aqueous lotion.

This invention also features the topical lotion composition according to the invention as pharmaceutical preparations useful to treat dermatological conditions/afflictions.

The formulation of ivermectin into a topical lotion composition for human use according to the invention is particularly useful for the treatment of rosacea, of common acne, of seborrhoeic dermatitis, of perioral dermatitis, of acneform rashes, of transient acantholytic dermatosis, and of acne necrotica miliaris.

The formulation of ivermectin into a topical lotion composition for human use according to the invention is more particularly useful in a regime or regimen for the treatment of rosacea.

### EXAMPLE 1

### Ivermectin Lotion

TABLE 1

| Sr. | | Quantity | Quantity | Quantity |
|---|---|---|---|---|
| No. | Ingredients | (% w/w) | (% w/w) | (% w/w) |
| 1 | Ivermectin | 1.00 | 2.00 | 3.00 |
| 2 | Isopropyl Alcohol | 38.00 | 38.00 | 38.00 |
| 3 | Carbomer | 1.50 | 1.50 | 1.50 |
| 4 | Purified Water | QS | QS | QS |

Process: Ivermectin was dissolved in isopropyl alcohol. Separately, carbomer was dispersed in water by high shear mixing. The ivermectin solution and carbomer dispersion were then mixed together until a medium viscosity lotion was formed. The lotion was filled and packed in bottles with a suitable spout cap.

What is claimed is:

1. A topical aqueous lotion composition comprising:
(a) Ivermectin;
(b) one or more pharmaceutically acceptable alcohols;
(c) one or more pharmaceutically acceptable gelling agents; and
(d) water;
wherein said lotion composition is devoid of surfactant.

2. The lotion composition of claim 1, wherein ivermectin is present in an amount of about 0.01% to about 5% by weight of said lotion composition.

3. The lotion composition of claim 1, wherein the pharmaceutically acceptable alcohol is present in an amount of at least 35% by weight of said lotion composition.

4. The lotion composition of claim 1, wherein the pharmaceutically acceptable gelling agent is present in an amount of at about 0.1% to about 5% by weight of said lotion composition.

5. The lotion composition of claim 1, wherein said alcohol is selected from the group consisting of methanol, benzyl alcohol, ethyl alcohol and isopropyl alcohol.

6. The lotion composition of claim 1, wherein the gelling agent is selected from the group consisting of hydroxypropyl cellulose, hydroxypropyl methylcellulose, carrageenans, microcrystalline cellulose, alginates, gellan gum, xanthan gum, veegum, hydroxyethyethylcellulose, guar gum, and carbomers.

7. The lotion composition of claim 1, wherein said lotion composition remains chemically and/or physically stable over a period of time of at least 8 weeks.

8. The lotion composition of claim 1, wherein said lotion composition further comprises one or more additives selected from the group consisting of flavour enhancers, preserving agents, stabilizers, humidity regulators, pH regulators, osmotic pressure modifiers, UV-A screening agents, UV-B screening agents and antioxidants.

9. The lotion composition of claim 1, wherein said lotion composition is devoid of preserving agents and antioxidants.

10. A method for the treatment of rosacea or inflammatory lesions of rosacea comprising topically applying the lotion composition of claim 1 on to the affected skin of the patient in need of such treatment.

11. A topical aqueous lotion composition comprising:
(a) Ivermectin;
(b) one or more pharmaceutically acceptable alcohols selected from the group consisting of methanol, benzyl alcohol, ethyl alcohol and isopropyl alcohol;
(c) one or more pharmaceutically acceptable gelling agents selected from the group consisting of hydroxypropyl cellulose, hydroxypropyl methylcellulose, carrageenans, microcrystalline cellulose, alginates, gellan gum, xanthan gum, veegum, hydroxyethyethylcellulose, guar gum, and carbomers; and
(d) water;
wherein said lotion composition is devoid of surfactant.

12. The topical aqueous lotion composition of claim 11, wherein said lotion composition is further devoid of preserving agents and antioxidants.

13. A topical aqueous lotion composition consisting essentially of:
(a) Ivermectin;
(b) one or more pharmaceutically acceptable alcohols comprising isopropyl alcohol;
(c) one or more pharmaceutically acceptable gelling agents comprising carbomer; and
(d) water;
wherein said lotion composition is devoid of surfactant.

14. The topical aqueous lotion composition of claim 13, consisting of:
(a) Ivermectin;
(b) isopropyl alcohol;
(c) carbomer; and
(d) water;
wherein said lotion composition is devoid of surfactant, preserving agents and antioxidants.

15. The topical aqueous lotion composition of claim 14, wherein the ivermectin is present in an amount of about 0.01% to about 5% by weight of said lotion composition.

16. The topical aqueous lotion composition of claim 14, wherein the isopropyl alcohol is present in an amount of at least 35% by weight of said lotion composition.

17. The topical aqueous lotion composition of claim 14, wherein the carbomers is present in an amount of at about 0.1% to about 5% by weight of said lotion composition.

18. The topical aqueous lotion composition of claim 13, consisting of:
(a) 1%-3% by weight of ivermectin;
(b) 38% by weight of isopropyl alcohol;
(c) 1.5% by weight of carbomer; and
(d) water.
